(19) United States
(12) Patent Application Publication
(10) Pub. No.: US 2015/0297384 A1
(43) Pub. Date: Oct. 22, 2015

(54) BRACE STRUCTURE

(71) Applicant: Tsan-Jee CHEN, Taipei City (TW)

(72) Inventor: Tsan-Jee CHEN, Taipei City (TW)

(21) Appl. No.: 14/258,811

(22) Filed: Apr. 22, 2014

(51) Int. Cl. A61F 5/01

(52) U.S. Cl. CPC A61F 5/0102

(57) ABSTRACT

A brace structure wearable at limb joints of a human operator is provided. The brace structure may include the central coverage area defined as an annular space by a circular knitted fabric object. A first adjustment band may extend from one end of the first stable area to be adhered to the first sticky surface. A second sticky surface and a second adjustment band may extend from one end of the second stable area to be adhered to the second sticky surface. The first stable area and the second stable area could be maintained at the upper end and the lower end of the limb joints when the first and second adjustment bands adhere to the sticky surfaces, so that the central coverage area could be maintained at the joints.

### BACKGROUND

1. Technical Field

The present disclosure relates to a brace structure, and more particularly, to a brace structure capable of adequately enclosing limb joints of a human operator to be covered.

2. Description of Related Art

The brace structure has been widely used in exercise or rehabilitation. Since joints generally adjoin limbs of the human operator, the joints are always required to be burdened with the pressure arising out of the movements of the limbs. Thus, the brace structure is there for the protection or relieving the pressure.

One of the conventional commercially available brace structure is made of a plurality of fabrics sewn together to be a barrel-shaped structure, in order to become wearable at limb joints of the human operator. When the limbs move, the joints need to shoulder the pressure associated with the bending and extending of limbs. When the limbs bend with respect to each other, the brace structure would be bent as well. As the conventional brace structure is inferior in its extend-ability because of the material it uses, the brace structure would not tightly enclose the limb joints to be covered. To remedy this, the traditional approach would be having an opening placed at the position where the limb joint to be covered locates, in order to enhance the extend-ability of the brace structure and ensure the brace structure tightly enclosing the surrounding skin of the limb joint to be covered.

However, with such opening the brace structure would not be effectively tightly enclosing the limb joint. The sliding of the brace structure around the joints would inevitably occur and therefore compromises the protection capability.

### SUMMARY OF THE DISCLOSURE

The brace structure according to the present disclosure is designed without the opening while still in possession of the sufficient extend-ability. Such brace structure could tightly enclose the joints for providing the adequate protection while minimizing the slipping of the brace structure with the installment of a first stable area and the second stable area.

The brace structure of the present disclosure may have its central coverage area made of circular knitted fabric object, which is used to enclose the joints to be covered, and such central coverage area could be offering the better permeability without compromising its performance in protecting the joints.

According to the present disclosure, a brace structure wearable at limb joints of a human operator is provided. Each of the limb joints referring to a joint and an upper end and a lower end of the joint. The brace structure may include the central coverage area defined as an annular space by a circular knitted fabric object. The first stable area may adjoin a first edge of the central coverage area, the first stable area may have a first intermediate layer made of a foamed material with an outer edge of the first intermediate layer having a first sticky surface, and a first adjustment band may extend from one end of the first stable area to be adhered to the first sticky surface. The second stable area may adjoin a second edge of the central coverage area, the second stable area may have a second intermediate layer made of a foamed material with an outer edge of the second intermediate layer having a second sticky surface and a second adjustment band may extend from one end of the second stable area to be adhered to the second sticky surface. Sticky tapes of the first stable area and the second stable area are fixed to the first sticky surface and the second sticky surface, respectively, allowing for the first stable area and the second stable area to be maintained at the upper end and the lower end of the joint, respectively, and enabling the central coverage area to tightly enclose the limb joint to be covered.

Specifically, the first adjustment band is configured to adjust the extent of the first stable area being maintained on the limb joints.

Specifically, the first stable area and the central coverage area are adjoined by high-frequency heat treatment or sewing.

Specifically, the second adjustment band is configured to adjust the extent of the second stable area being maintained on the limb joints.

Specifically, the second stable area and the central coverage area are adjoined by high-frequency heat treatment or sewing.

Specifically, the central coverage area is 1-4 mini-meters in thickness.

Specifically, the circular knitted fabric object is formed because of bending of threads of yarn in form of coils, when the coils longitudinally stretch the width of the coils reduces with the height of the coils increasing and when the coils are laterally stretched the width of the coils increases with the height of the coils decreasing, rendering possible the circular knitted fabric object extending in all directions.

For further understanding of the present disclosure, reference is made to the following detailed description illustrating the embodiments and examples of the present disclosure. The description is only for illustrating the present disclosure, not for limiting the scope of the claim.

### BRIEF DESCRIPTION OF THE DRAWINGS

The drawings included herein provide further understanding of the present disclosure. A brief introduction of the drawings is as follows:

FIG. 1 shows a schematic diagram of a brace structure according to one embodiment of the present disclosure;

FIG. 2 shows another schematic diagram of a brace structure according to one embodiment of the present disclosure;

FIG. 3A shows a cross-sectional view of a first stable area of the brace structure according to one embodiment of the present disclosure;

FIG. 3B shows a cross-sectional view of a second stable area of the brace structure according to one embodiment of the present disclosure;

FIG. 4 shows a schematic diagram of using the brace structure to protect the knee joint according to one embodiment of the present disclosure;

FIG. 5 is a schematic diagram of using the brace structure to protect the elbow joint according to one embodiment of the present disclosure;

FIG. 6 is a schematic diagram of using the brace structure to protect the wrist joint according to one embodiment of the present disclosure; and

FIG. 7 is a schematic diagram of using the brace structure to protect the ankle joint according to one embodiment of the present disclosure.

### DESCRIPTION OF THE EXEMPLARY EMBODIMENTS

The aforementioned and other technical contents, features, and efficacies will be shown in the following detail descriptions of a preferred embodiment corresponding with the reference Figures.

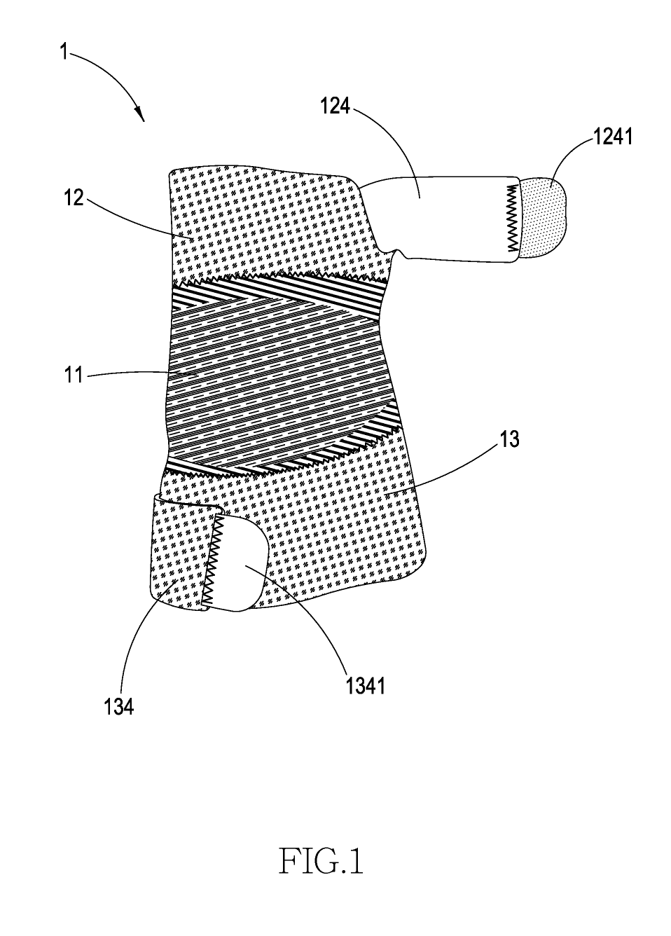

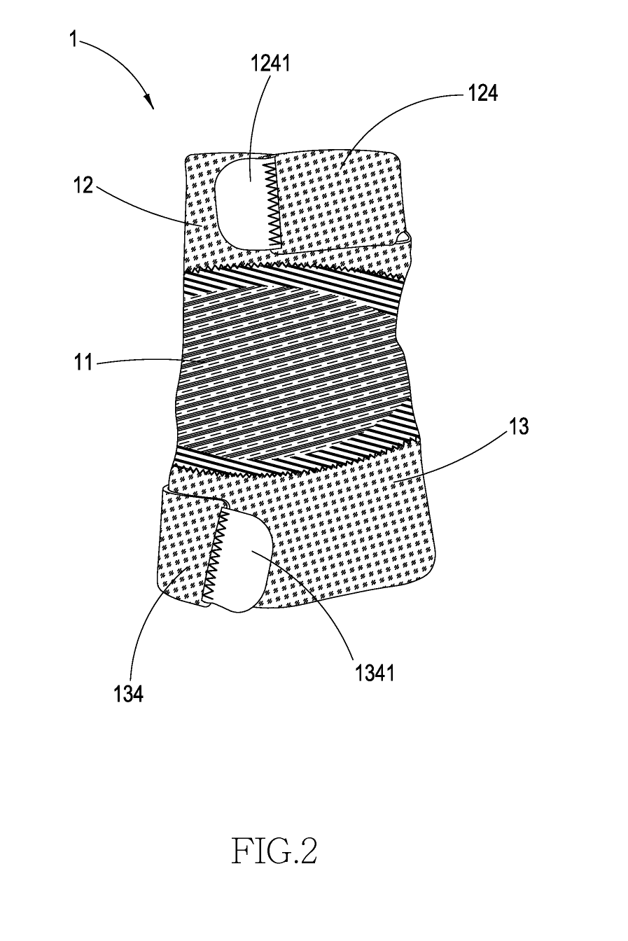

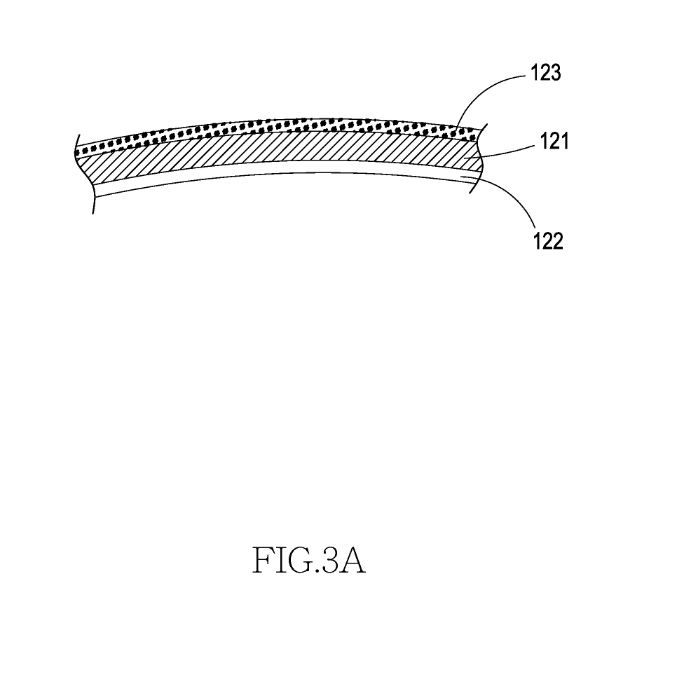

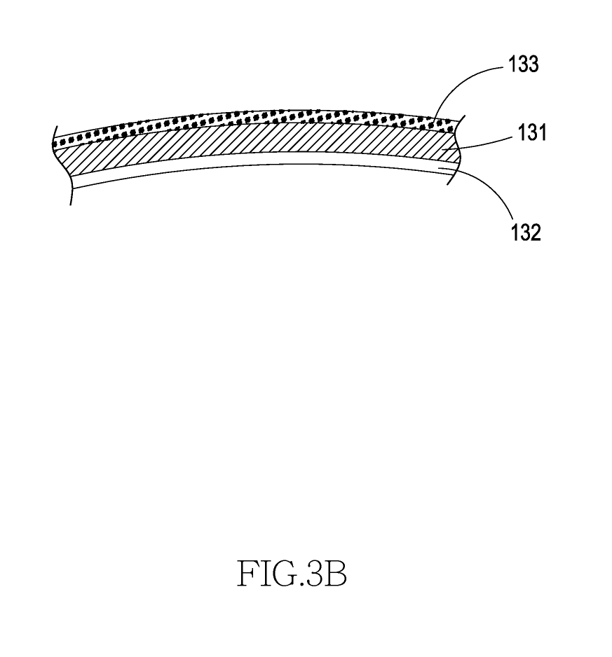

Please refer to FIGS. 1-3 of schematic diagrams of a brace structure and a first stable area of the brace structure according to one embodiment of the present disclosure. The brace structure may include a central coverage area 11, a first stable area 12 and a second stable area 13. In one implementation, the central coverage area 11 may be defined as an annular space and made of a circular knitted fabric object. The circular knitted fabric object may be formed because of bending of threads of yarn in form of coils. Each coil may be formed by a thread of yarn and when the coils longitudinally stretch the width of the coils reduces with the height of the coils increasing. And when the coils are laterally stretched the width of the coils increases with the height of the coils decreasing. The width and the height of the coils may vary depending on different stretching, so that the circular knitted fabric object may extend in all directions because of its flexibility. When the circular knitted fabric object is formed by the threads of the yarn in form of the coils, better permeability and sense of may be obtained, enabling the better coverage of the central coverage area 11 enclosing limb joints to be covered.

The first stable area 12 and the second stable area 13 may be adapted to be maintained at portions of the limb joints when in use. More specifically, the first stable area 12 and the second stable area 13 may be maintained at an upper end and a lower end of a joint to be covered. Thus, the central coverage area 11 may be used to be maintained at the area between the upper end and the lower end the joint. The limb joint in the present disclosure may refer to the limb and the upper end and the lower end thereof collectively. The first stable area 12 and the second stable area 13 may adjoin different edges of the central coverage area 11. Such adjoining may be implemented by the high-frequency heat treatment or sewing. The first stable area 12 and the second stable area 13 may be having a first intermediate layer 121 and a second intermediate layer 131, respectively. Both the first intermediate layer 121 and the second intermediate layer 131 may be made of a foamed material. An inner edge 122 or 132 of the first intermediate layer 121 or the second intermediate layer 131 may be a non-slippery in nature, while an outer edge of the first intermediate layer 121 or the second intermediate layer may be associated with a sticky surface. For example, the first intermediate layer 121 may have a first sticky surface 123 and the second intermediate layer 131 may have a second sticky surface 133.

An adjustment band may extend from one end of the first stabling area 12 or the second stabling area 13. In one implementation, a first adjustment band 124 may extend from one end of the first stabling area 12, while a second adjustment band 134 may extend from one end of the second stabling area 13. The first adjustment bend 124 and the second adjustment bend 134 may be having sticky tapes 1241 and 1341, respectively. When the brace structure 1 is wore by any human operator, the sticky tapes 1241 and 1341 may be adhered to the first sticky surface 123 and the second sticky surface 133 respectively to adjust the extent of the first stable area 12 and the second stable area 13 being maintained on the upper end and the lower end of the limb joint. By adjusting the extent of both the first stable area 12 and the second stable area 13 being maintained on the limb joint, the brace structure 1 may end up adjusting the extent of the central coverage area 11 being maintained on the area between the lower end and the upper end of the limb joint.

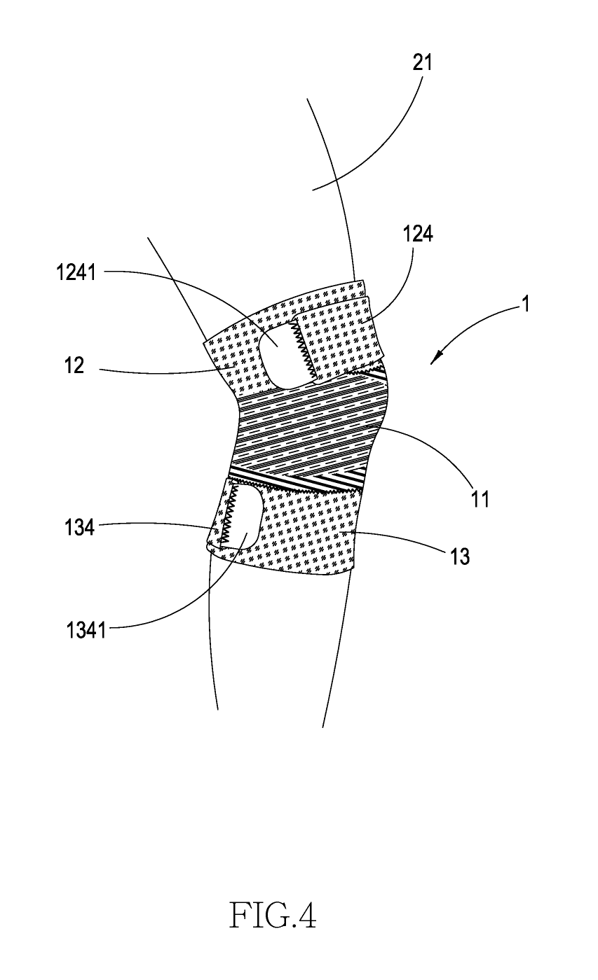

When the limb joint to be covered or enclosed by the central coverage area 11 of the brace structure 1 is the knee joint, as shown in FIG. 4, and when the human operator wears the brace structure 1 on his/her leg 21, the central coverage area 11 may be placed on the knee joint of the leg 21 first. Thereafter, the extent of the central coverage area 11 to be maintained on the knee joint may be adjusted by having the sticky tapes 1241 and 1341 adhered to the first sticky surface 123 and the second sticky surface 133, which may adjust the extent of the first stable area 12 and the second stable area 13 to be maintained at the upper end and the lower end of the knee joint.

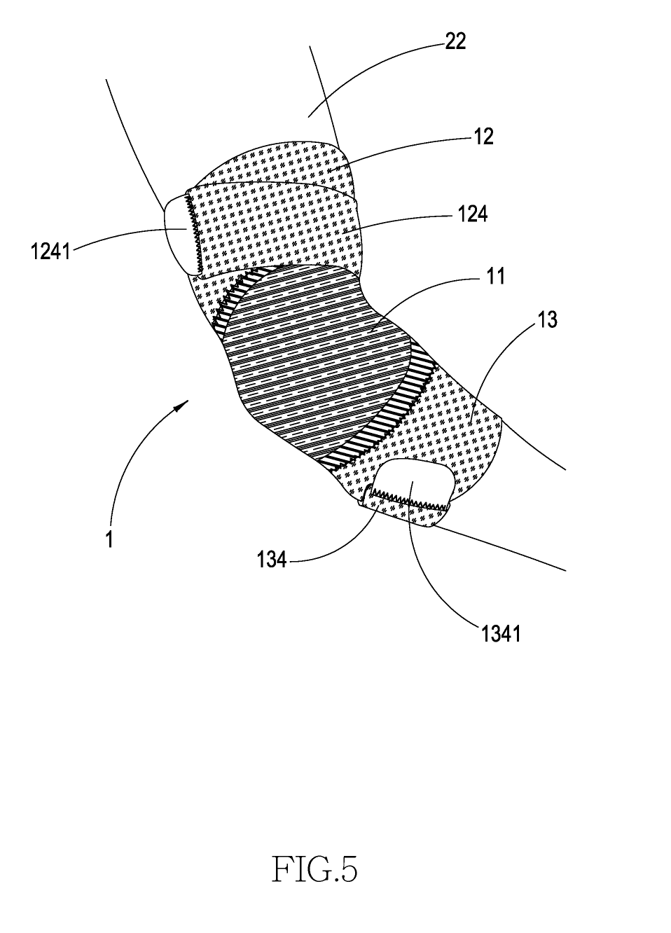

When the limb joint to be covered or enclosed by the central coverage area 11 of the brace structure 1 is the elbow joint, as shown in FIG. 5, and when the human operator wears the brace structure 1 on his/her arm 22, the central coverage area 11 may be placed on the elbow joint of the arm 22 first. Thereafter, the extent of the central coverage area 11 to be maintained on the elbow joint may be adjusted by having the sticky tapes 1241 and 1341 adhered to the first sticky surface 123 and the second sticky surface 133, which may adjust the extent of the first stable area 12 and the second stable area 13 to be maintained at the upper end and the lower end of the elbow joint.

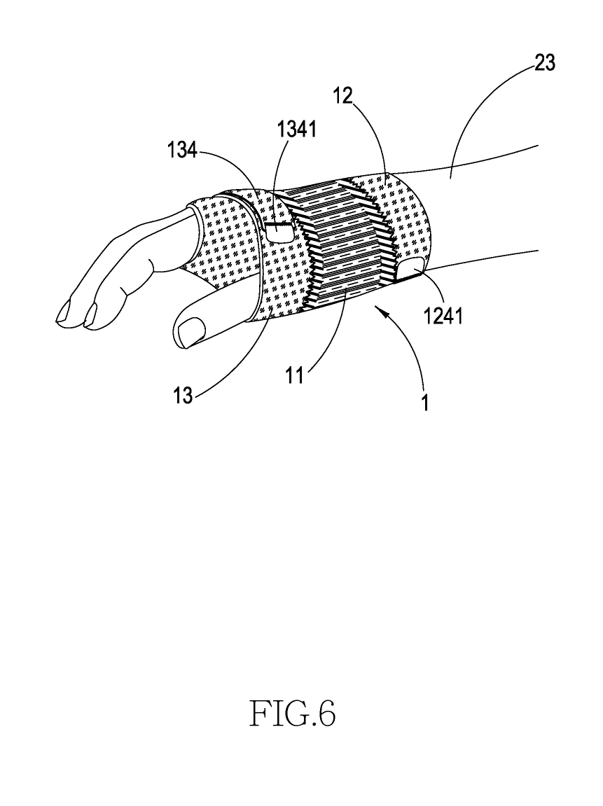

When the limb joint to be covered/enclosed by the central coverage area 11 of the brace structure 1 is the wrist joint, as shown in FIG. 6, and when the human operator wears the brace structure 1 on his/her forearm 23, the central coverage area 11 may be placed on the wrist joint of the forearm 23 first. Thereafter, the extent of the central coverage area 11 to be maintained on the wrist joint may be adjusted by having the sticky tapes 1241 and 1341 adhered to the first sticky surface 123 and the second sticky surface 133, which may adjust the extent of the first stable area 12 and the second stable area 13 to be maintained at the upper end and the lower end of the wrist joint.

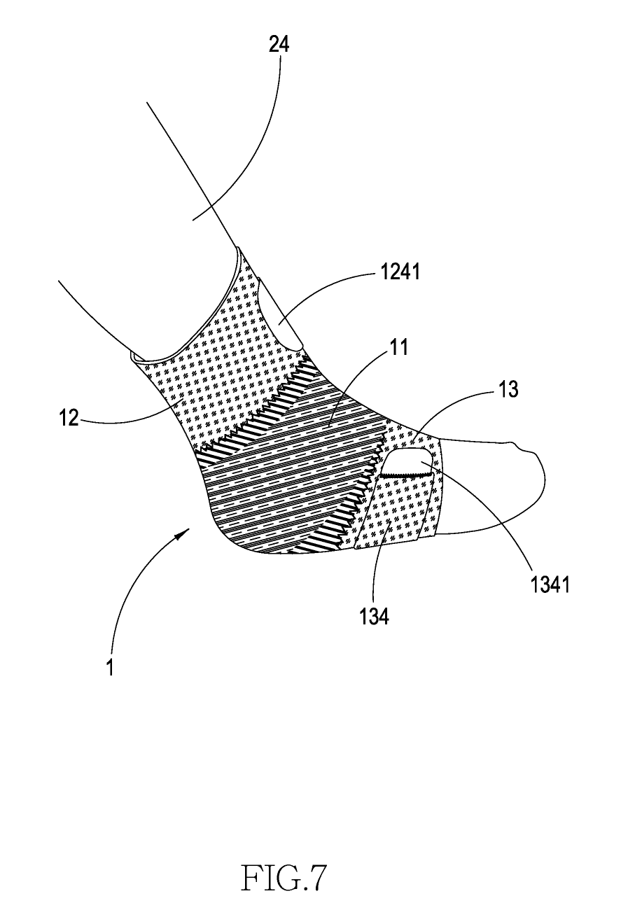

When the limb joint to be covered or enclosed by the central coverage area 11 of the brace structure 1 is the ankle joint, as shown in FIG. 7, and when the human operator wears the brace structure 1 on his/her calf 24, the central coverage area 11 may be placed on the ankle joint of the calf 24 first. Thereafter, the extent of the central coverage area 11 to be maintained on the ankle joint may be adjusted by having the sticky tapes 1241 and 1341 adhered to the first sticky surface 123 and the second sticky surface 133, which may adjust the extent of the first stable area 12 and the second stable area 13 to be maintained at the upper end and the lower end of the ankle joint.

When compared with other conventional techniques, the present disclosure may offer the following advantage: (1) the brace structure requires no opening corresponding to the limb joint to be covered or enclosed and may be in possession of the sufficient amount of extend-ability to properly enclose the limb joint to be covered without too much undesired sliding because of the limb movement/activity, which could offer the proper protection for the limb joint, (2) that the central coverage area is made of circular knitted fabric object could properly render possible the enclosing of the limb joint to be covered while providing the adequate protection for the limb joint in addition to offering the better permeability for the same, and (3) with the adjustment bands the brace structure according to the present disclosure may enable the extent of the brace structure to enclose the limb joint to be adjusted with ease.

Some modifications of these examples, as well as other possibilities will, on reading or having read this description, or having comprehended these examples, will occur to those skilled in the art. Such modifications and variations are comprehended within this disclosure as described here and claimed below. The description above illustrates only a relative few specific embodiments and examples of the present disclosure. The present disclosure, indeed, does include various modifications and variations made to the structures and operations described herein, which still fall within the scope of the present disclosure as defined in the following claims.

What is claimed is:

1. A brace structure wearable at limb joints of a human operator, each of the limb joints referring to a joint and an upper end and a lower end of the joint, the brace structure comprising:
a central coverage area defined as an annular space by a circular knitted fabric object;
a first stable area adjoining a first edge of the central coverage area, the first stable area having a first intermediate layer made of a foamed material with an outer edge of the first intermediate layer having a first sticky surface, and a first adjustment band extending from one end of the first stable area to be adhered to the first sticky surface; and
a second stable area adjoining a second edge of the central coverage area, the second stable area having a second intermediate layer made of a foamed material with an outer edge of the second intermediate layer having a second sticky surface and a second adjustment band extending from one end of the second stable area to be adhered to the second sticky surface;
wherein sticky tapes of the first stable area and the second stable area are fixed to the first sticky surface and the second sticky surface, respectively, allowing for the first stable area and the second stable area to be maintained at the upper end and the lower end of the joint, respectively, and enabling the central coverage area to tightly enclose the limb joint to be covered.

2. The brace structure according to claim 1, wherein the first adjustment band is configured to adjust the extent of the first stable area being maintained on the limb joints.

3. The brace structure according to claim 1, wherein the first stable area and the central coverage area are adjoined by high-frequency heat treatment or sewing.

4. The brace structure according to claim 1, wherein the second adjustment band is configured to adjust the extent of the second stable area being maintained on the limb joints.

5. The brace structure according to claim 1, wherein the second stable area and the central coverage area are adjoined by high-frequency heat treatment or sewing.

6. The brace structure according to claim 1, wherein the central coverage area is 1-4 mini-meters in thickness.

7. The brace structure according to claim 1, wherein the circular knitted fabric object is formed because of bending of threads of yarn in form of coils, when the coils longitudinally stretch the width of the coils reduces with the height of the coils increasing and when the coils are laterally stretched the width of the coils increases with the height of the coils decreasing, rendering possible the circular knitted fabric object extending in all directions.
